(19) United States
(12) Patent Application Publication
(10) Pub. No.: US 2023/0260666 A1
(43) Pub. Date: Aug. 17, 2023

(54) SYSTEMS AND METHODS FOR BLIND SPOT TRACKING

(71) Applicant: Thomas Jefferson University, Philadelphia, PA (US)

(72) Inventors: James J. Evans, Philadelphia, PA (US); Nikolaos Mouchtouris, Philadelphia, PA (US); Vadim Geyfman, Marlton, NJ (US)

(21) Appl. No.: 18/012,399

(22) PCT Filed: Jun. 25, 2021
(86) PCT No.: PCT/US21/39138

§ 371 Date: Dec. 22, 2022

Related U.S. Application Data
(60) Provisional application No. 63/044,130, filed on Jun. 25, 2020.

(51) Int. Cl. G16H 80/00; A61B 3/00; A61B 3/032

(52) U.S. Cl. CPC G16H 80/00; A61B 3/0033; A61B 3/032

(57) ABSTRACT

Systems and methods for blind spot tracking are described herein. In one aspect, a computer-implemented method can include generating a repositionable animated object on a display screen; receiving input from the user of the display screen when the repositionable animated object transitions from within a viewing range of the user to outside of the viewing range; determining a position of the repositionable animated object on the display screen based on a timing of the received input; and determining a distance away from the display screen for the user based on the position of the repositionable animated object.

### CROSS-REFERENCE TO RELATED APPLICATION(S)

This application claims priority to U.S. Provisional Patent Application No. 63/044,130 filed Jun. 25, 2020, which is incorporated herein by reference in its entirety.

### BACKGROUND OF THE INVENTION

Conventional medical practices are often limited to in-person meetings between a patient and a medical professional. This can be a great burden on a patient, particularly where the patient lives a significant distance away from a corresponding medical center, or if the patient's medical condition requires numerous patient-medical professional interactions.

Telemedicine offers the ability to reduce these patient burdens. However, while advances have been made in telemedicine, conventional telemedicine platforms are limited in their ability to perform certain examinations. For example, conventional telemedicine platforms are limited in performing neuro-ophthalmic examinations, as these platforms rely on a static distance between the patient and a telemedicine screen (e.g., a phone, tablet, computer, and the like) to conduct the examination. However, these static distance examinations are unable to account for variation in a patient's blind spot, and are at risk for significant systematic error.

### SUMMARY

Systems and methods for blind spot tracking are described herein. In one aspect, a computer-implemented method can include generating a repositionable animated object on a display screen; receiving input from the user of the display screen when the repositionable animated object transitions from within a viewing range of the user to outside of the viewing range; determining a position of the repositionable animated object on the display screen based on a timing of the received input; and determining a blind spot of the user on the display screen based on the position of the repositionable animated object.

This aspect can include a variety of embodiments. In one embodiment, the computer-implemented method can further include repositioning the repositionable animated object in a predefined direction of the screen and at a predefined speed. In some cases, the predefined direction can include a lateral direction, and the blind spot can include a lateral blind spot. In some cases, the predefined direction includes a vertical direction, and the blind spot can include a lateral blind spot.

In another embodiment, the computer-implemented methods can further include generating a neuro-ophthalmic test for the user based on a location of the blind spot on the display screen and a distance away from the display screen of the user. In some cases, the method can further include generating an object size, an object color, an object luminosity, or an object graphic positioning of the neuro-ophthalmic test according to the determined distance away from the display screen for the user. In some cases, the neuro-ophthalmic test can include a pituitary adenoma test, a brain tumor test, a multiple sclerosis test, a neuromyelitis optica test, an optic neuritis test, an ischemic optic neuropathy test, a giant cell arteritis test, an optic neuropathy test, a retinal degenerations test, a toxic optic neuropathies and retinopathies test, or a combination thereof.

In another embodiment, the computer-implemented can further include generating a static reference point on the display screen, where a repositioning of the repositionable animated object occurs in relation to the static reference point.

In another embodiment, a telemedicine system can include a user device including at least the display screen; and a processor configured to perform the computer-implemented method.

In another aspect, a computer-implemented method for identifying a blind spot of a user includes: generating a repositionable animated object on a display screen; receiving input from the user of the display screen when the repositionable animated object transitions from within a viewing range of the user to outside of the viewing range; determining a position of the repositionable animated object on the display screen based on a timing of the received input; and determining at least one blind spot for the user based on the position of the repositionable animated object.

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

FIG. 1 depicts a system for blind spot tracking according to an embodiment of the present disclosure.

FIG. 2 depicts a server for blind spot tracking procedures according to an embodiment of the present disclosure.

FIG. 3A and 3B depict a blind spot tracking procedure according to an embodiment of the present disclosure.

FIG. 4 depicts an Amsler grid examination according to an embodiment of the present disclosure.

FIG. 5A and 5B depict a kinetic blind spot tracking procedure according to an embodiment of the present disclosure.

FIG. 6 depicts a blind calibration procedure according to an embodiment of the present disclosure.

FIG. 7 depicts a visual acuity testing procedure according to an embodiment of the present disclosure.

FIG. 8 depicts a static field testing procedure according to an embodiment of the present disclosure.

FIG. 9 depicts a kinetic visual field testing procedure according to an embodiment of the present disclosure.

FIG. 10 depicts a blind spot-based Amsler grid assessment procedure according to an embodiment of the present disclosure.

FIG. 11 depicts static visual fields (Panel A) and kinetic visual fields (Panel B) demonstrating severely constricted peripheral vision with visual field deficits in bilateral superior temporal quadrant in both eyes for a patient with a history of pituitary adenoma and having bilateral superior temporal visual field deficits. Each dot represents a location where the user observed the dot while focusing on the axis intersection. The shade of the dot represents the time period before the user indicated he/she observed that particular dot (with darker equating to a longer time period).

FIG. 12 depicts static visual fields (Panel A) and kinetic visual fields (Panel B) demonstrating full visual fields in both eyes for a patient with a history of pituitary adenoma.

### DEFINITIONS

The instant invention is most clearly understood with reference to the following definitions.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

### DETAILED DESCRIPTION OF THE INVENTION

Systems and computer-implemented methods for blind spot tracking are described herein. A telemedicine system can utilize a user's blind spot to calculate the distance of the user from a computer screen and can administer a series of customized, personalized neuro-ophthalmic tests based on the distance from the screen. Using each person's specific blind spots allows for standardized and reliable testing of a patient's visual acuity and peripheral vision. For example, the font size of a visual acuity test can be adjusted based on the blind-spot calibration in order to obtain accurate results regardless of the patient's position. Static and kinetic visual perimetry tests can also be modified based on the blind spot to capture wider angles of peripheral vision than previously feasible using a conventional computer screen. The system can further record each patient's baseline vision characteristics and/or pre-existing deficits and can focus subsequent examinations on the areas of interest.

The system and computer-implemented methods described herein can provide a personalized patient assessment without requiring the presence of a healthcare provider or technician to conduct the examination. This way, untrained users can undergo the assessment at home. In a study of 11,449 patients, Dr. Wang et al demonstrated significant variability for a patient in the blind spot location in both the horizontal and vertical orientations. The coefficient of variation in the horizontal and vertical orientations was found to be 9.5% and 62.1% respectively (Wang, M., Shen, L. Q., Boland, M. V., Wellik, S. R., De Moraes, C. G., Myers, J. S., . . . Elze, T. (2017). Impact of Natural Blind Spot Location on Perimetry. Sci Rep, 7(1), 6143. doi: 10.1038/s41598-017-06580-7). Therefore, conventional telemedicine platforms that offer static neuro-ophthalmic testing at a fixed distance from the screen are unable to account for the variation in each patient's blind spot(s) and are at risk for significant systematic error. Further, these conventional platforms are unable to account for pre-existing deficits as well as user error, all of which can compromise results. The disclosure described herein addresses these limitations by adjusting the size, color, luminosity, and spatial relationships of each neuro-ophthalmic test based on the user's blind spot calibration, thereby minimizing user error. The systems described herein can further monitor patients with ocular diseases such as glaucoma, and neurological diseases such as pituitary adenomas and other brain tumors, and remotely track their progress before and after treatment.

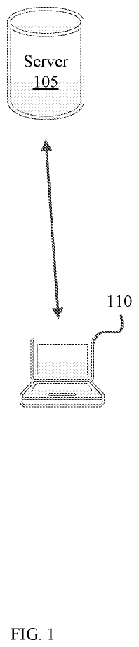

FIG. 1 depicts a system for blind spot tracking according to an embodiment of the present disclosure. The system can include a server 105 and a computing device 110.

The server 105 can store instructions for performing a blind spot tracking procedure. In some cases, the instructions for performing a blind spot tracking procedure can be relayed to the computing device 110 for execution, and in some cases the instructions can be downloaded by the computing device 110 (e.g., stored locally). In some cases, the server 105 can also include a set of processors that execute the set of instructions. Further, the server 105 can be any type of server capable of storing and/or executing instructions, for example, an application server, a web server, a proxy server, a file transfer protocol (FTP) server, and the like. In some cases, the server 105 can be a part of a cloud computing architecture, such as a Software as a Service (SaaS), Development as a Service (DaaS), Data as a Service (DaaS), Platform as a Service (PaaS), and Infrastructure as a Service (IaaS).

A computing device 110 can be in electronic communication with the server 105 and can display the blind spot tracking procedure to a user. The computing device 110 can include a display for displaying the blind spot tracking procedure, and a user input device, such as a mouse, keyboard, or touchpad, for logging and transmitting user input corresponding to the blind spot tracking procedure. In some cases, the computing device 110 can include a set of processors for executing the blind spot tracking procedure (e.g., from instructions stored in memory). Examples of a computing device include, but are not limited to, a personal computer, a laptop, a tablet, a cellphone, a personal digital assistant, an e-reader, a mobile gaming device, and the like.

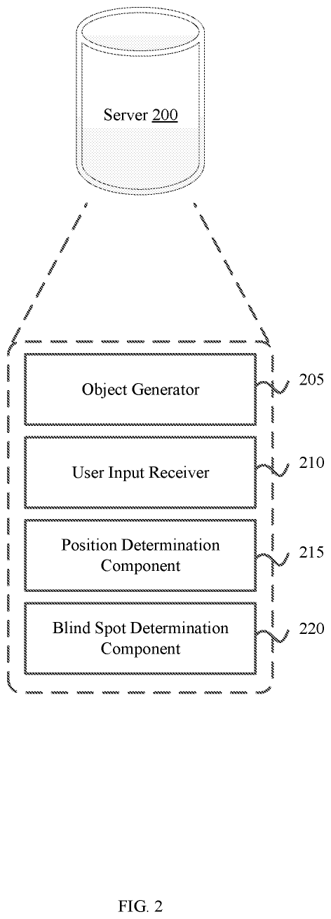

FIG. 2 depicts a server 200 for blind spot tracking according to an embodiment of the present disclosure. The server can be an example of the server 105 as discussed with reference to FIG. 1. The server 200 can include an object generator 205, a user input receiver 210, an object position determination component 215, and a user distance determination component 220.

The object generator 205 can generate a repositionable animated object for the display screen of the computing device of a user, such as the computing device 110 as described with reference to FIG. 1. The repositionable animated object can be any number of objects having a defined body, which includes but is not limited to a dot, a circle, a triangle, a star, a rectangle, an ellipse, and the like. Further, the object generator 205 can reposition the animated object on the display screen over a period of time. For example, the animated object can move in a predefined direction at a predefined speed across the display upon initiation of the blind spot tracking procedure. In some cases, the object generator can also generate a reference point to be displayed by the display. The reference point may be a stationary object displayed on the screen. In some cases, the animated object may move in relation to the reference point, for example moving away from, or towards the reference point.

The user input receiver 210 can receive user input from the computing device. For example, the user input can be a mouse click, a keyboard click, a touch on a touchpad, and the like. The user input receiver 210 can receive the user input and log different parameters of the user input. For example, the user input receiver 210 can identify a timestamp of the user input, the type of user input (e.g., mouse click, keyboard click, etc.) and the like. The server 200 may store the user input in memory.

The position determination component 215 can determine a position of the animated object based on the received user input. As discussed above, the animated object may be repositioned on the display screen during the blind spot tracking procedure. The position determination component 215 can determine the position of the animated object at the time the user provides input via the computing device. The determination can be based on a timestamp of the received user input. In some cases, the determination can be based on the predefined speed, the predefined direction, and/or an initiation timestamp corresponding to when the blind spot tracking procedure began (e.g., when the animated object initiated movement on the display).

The blind spot determination component 220 can determine the a blind spot location on the display screen for the user based on the determined position of the animated object. The blind spot determination component 220 can determine a distance away from the reference point for the animated object when the user provides user input. In some cases, the distance determination component can further determine the distance between an end position of the animated object and an initial position of the animated object based on the received user input. For example, the distance between the reference point and the position of the animated object as determined by the received user input can be calculated and converted to angular degrees. The vector connecting the reference point and the animated object can be normal to the vector connecting the reference point and the user. Using the relationship and length of these two vectors, the distance between the reference point and the user is thereby calculated.

For example, a user's blind spot is considered to be 10-15 degrees from the focal point of focus based on population-based studies on blind spots. By identifying the blind spots of a user on a display screen, the system can identify the pixel location of the user's indicated blind spot, and convert the pixel location to angular degrees. The angular degrees can, in conjunction with the knowledge of the 10-15 degree location of a general user's blind spot, the system can adjust the neuro-ophthalmology test displayed to the user based on the determined angular degrees. For the ophthalmologic assessments, animated objects appear at different points in the screen in a random order. When the user identifies these objects, their location is recorded and their distance from the focus point is determined in angular degrees.

Many neuro-ophthalmology tests rely on angular degrees of a user's field of vision for implementation and/or user's vision measurements. In particular: an Amsler grid is a test that measures the central 20 degrees of visual field; a Snellen chart used for the Visual Acuity test is sized so that the letters on the 20/20 line subtend 5 minutes of arc (1/12th of 1 degree); a static visual fields test is sized to fill the available screen space, and the location of the visual stimuli is spaced out in whole degree increments relative to the focal point of focus of the user on the screen; and a kinetic visual fields test result is measured in degrees relative to the focal point.

### Use Example

A user can access a telemedicine website or portal which is in electronic communication with a server, such as server 105 or 200 of FIGS. 1 and 2, respectively. In some cases, the user will be prompted for identification credentials (e.g., username, password, etc.) prior to gaining access. The user may have the option of selecting a telemedicine exam he/she wishes to undertake. In some cases, the user may be provided with instructions on how to properly participate in the selected telemedicine exam.

The user may then be shown a screen with an animated object. The animated object may begin moving on the screen in a predefined direction and a predefined speed. Further, the user may be instructed to focus his/her eyesight on a particular location of the display. For example, the user may be instructed to focus his/her eyesight on a reference point that remains statically positioned during the blind spot tracking procedure. The user may be further instructed to provide input when the animated object moves to a position where the user can no longer view the animated object (e.g., when the animated object enters a blind spot for the user). In some cases, the user may be instructed to also provide input when the animated object moves back into the user's eyesight.

Once the user provides input, the system may generate a verification procedure corresponding to the user inputs received for the blind spot tracking procedure. For example, the verification procedure may include at least one animated object in a determined position on the display. The determined position may correspond to a determined blind spot location for the user at the user's distance away from the display and the reference point of where the user is instructed to look. The user may be instructed to verify that the animated object at the predefined position is not in the eyesight of the user while the user is focused on the reference point. In some cases, the verification procedure may include multiple animated objects, each at a determined location on the display.

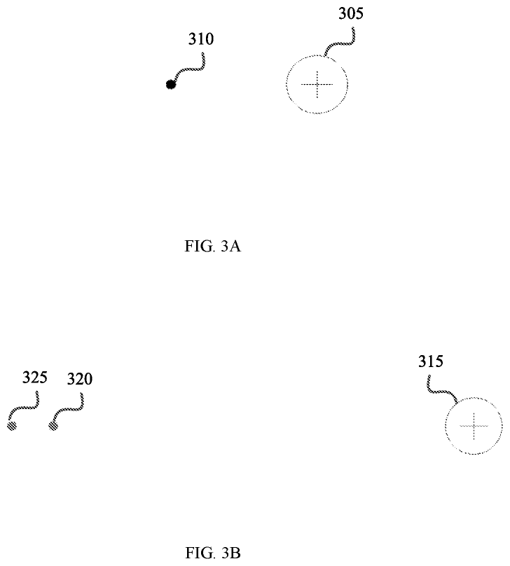

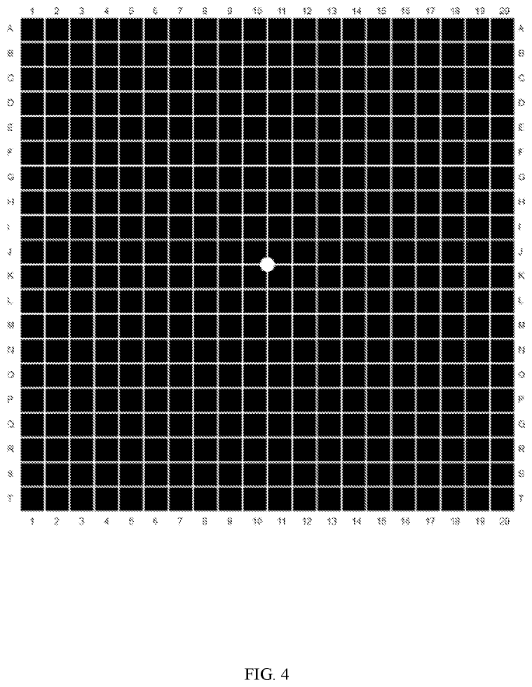

FIG. 3A and 3B depict screenshots of a blind spot tracking procedure according to embodiments of the present disclosure. In FIG. 3A, the user may be instructed to focus on the reference point 305, which may be statically positioned. The animated object 310 may reposition on the display at a predefined speed and direction. In this case, the animated object 310 may move towards and away from the reference point 305. The user is instructed to focus on the reference point 305 while the animated object 310 moves away from the reference point 305. The user is further instructed to provide input when the animated object 310 is no longer within the user's eyesight as the user focuses on the reference point 305. The user is further instructed to provide input again once the animated object 310 returns to the user's eyesight (as the animated object 310 continues to move away from the reference point 305). After user input is provided, the system may implement a verification procedure. FIG. 3B depicts a screenshot of a verification procedure according to embodiments of the claimed invention. The verification procedure in this case includes a reference point 315 and animated objects 320 and 325. The user is instructed to focus his/her eyesight (e.g., either the left or right eye) on the reference point 315. The user is further instructed to either affirm or deny the animated objects 320 and 325 are out of the user's eyesight (e.g., within the user's blind spot) while the user focuses on the reference point 315. The user can provide additional input to either affirm or deny the animated objects 320 and 325 are outside of the user's eyesight.

The system can then determine the distance away the user is from the display. The system can identify the position of the user's blind spot, along with the relative size of the user's blind spot, from the user input, the location of the animated object, the speed and direction the animated object is traveling, and the like.

The system can then modify other components of neuro-ophthalmic examinations based on the determined distance between the user and the screen. Neuro-ophthalmic examinations can include, for example, assessments for retinal degeneration, giant cell arteritis, ischemic optic neuropathy, pituitary adenoma, brain tumors, multiple sclerosis, neuromyelitis optica, optic neuritis, cone-rod dystrophy, toxic optic neuropathies or retinopathies, and the like. For example, font size, font color, object size, object color, object luminosity, object positioning, and the like, of a neuro-ophthalmic examination can be modified according to a user's distance away from the screen. As these types of neuro-ophthalmic examinations heavily rely on standard formatting for implementation, modifying different characteristics of the examination can minimize user error and inaccurate results. For example, FIG. 4 depicts an Amsler grid according to embodiments of the present disclosure. An Amsler grid can be used as part of a macular degeneration test. The Amsler grid in FIG. 4 can be modified based on the determined distance away from the screen a user is, for example, the dimensions of the grid can be modified. The user interprets the pattern and angles of the lines displayed in the grid, which allows for the evaluation and detection of macular degeneration.

In one example, a user's visual acuity can be assessed using examination procedures dependent on (e.g., calibrated by) the user's determined distance away from the screen. A row of letters can be displayed to the user. The initial sizing of the letters, and the sizing of subsequent letters, can be determined according to the user's determined distance away from the screen. The user may be asked to read the displayed letters out loud. Using voice recognition capabilities, a new row of different letters in a different (e.g., smaller) font size can be displayed to the user once the user provides the identity of the preceding displayed letters. When the user is unable to correctly identify the displayed letters anymore, the font size of that row can be stored in memory. Subsequently, the user's visual acuity can be determined based on the letter size(s) the user is able to identify or not identify.

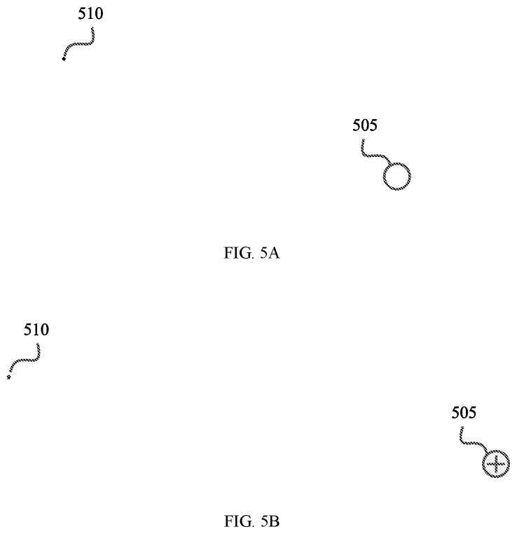

Another blind spot tracking procedure is depicted in FIG. 5A and 5B. In this example of a blind spot tracking procedure, a user is instructed to focus on the reference point 505 (e.g., with either the left or right eye). The animated object 510 may move towards the reference point 505 at a predefined speed from a predefined direction. The user may be instructed to provide user input when the animated object 510 is inside the eyesight of the user as the user focuses on the reference point 505. Once the user provides this input, the reference point 505 may be repositioned, as well as the animated object 5(as depicted in FIG. 5B). The animated object 510 may move towards the reference point 505 at a different speed and direction compared to the preceding movement (depicted in FIG. 5A). This procedure allows for efficient peripheral vision examination with improved detection of the shape and edges of visual field deficits. The efficient assessment of the user's visual fields using the kinetic perimetry is used to generate a patient-specific and focused assessment of the user's visual field in question.

### Experimental Data

As health systems expand their catchment areas, patients find themselves at an increasing distance from their neurosurgical care centers. Patients and their family members are often burdened by the long travel and wait times and may even be required to take multiple days off of work to visit the doctor. While telemedicine is growing to address the needs of patients in resource-limited areas, current telemedicine platforms are still limited in enabling physicians to perform a physical exam online; specifically, platforms lack a solution for performing neuro-ophthalmic testing, a necessary component of examination after brain tumor surgery.

This pressing need for telemedicine technology that is specific for patients with neurological disorders became even more evident with the COVID-19 pandemic. As the lockdown measures were put in place, access to healthcare became a challenge. There was a surge in telemedicine use; however, obtaining a neurological exam still necessitated an in-person visit. As a result, patients chose to either not seek care or to put themselves at risk.

### Methods

The software implementing the techniques described herein allows users to undergo neuro-ophthalmic testing using a blind spot-based calibration technique that accounts for the patient's distance from the screen and ensures the reliability of neuro-ophthalmic test results.

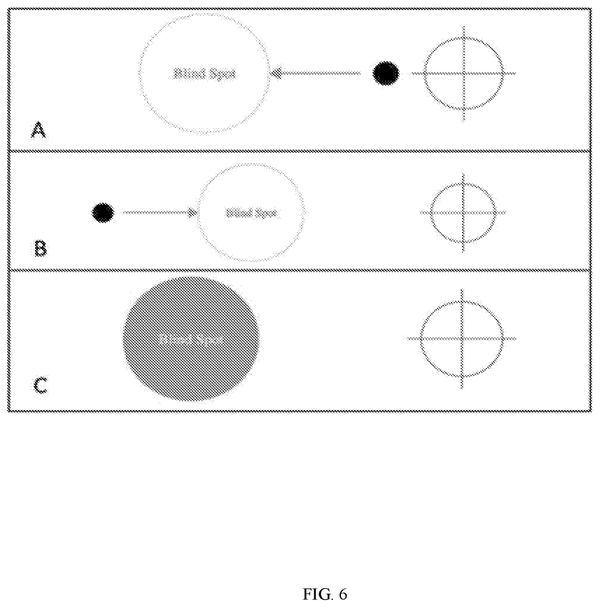

The assessment starts with determining the user's blind spot by asking the user to close the right eye and focus with the left eye on a crosshair that is located on the right end of the screen (FIG. 6A). A moving dot appears and moves from the crosshair across the screen (FIG. 6A). The user is asked to press a button on the keyboard as soon as they stop seeing the moving dot. At that point, the dot has entered the right border of the blind spot and the user is unable to see the dot. Then, the dot reappears from the left end of the screen and starts moving towards the crosshair (FIG. 6B). The user is asked to press a button on the keyboard when the dot disappears. The point at which the dot disappears delineates the left-sided border of the user's blind spot. Once that is completed, the user is asked to focus with the left eye on the crosshair one more time, and a blinking circle is displayed at the blind spot (FIG. 6C). The user should not be able to see the blinking dot. If that is the case, the blind spot calibration has been performed correctly and the user can proceed with the assessments. The same process is repeated with the left eye closed and the right eye focusing on a crosshair located on the left end of the screen.

The distance in pixels between the crosshair and the user's blind spot is determined and is used to determine how far the user is sitting from the screen. Based on the user's distance from the computer, the software adjusts the font size and spatial relationships of each the following neuro-ophthalmic assessments in order to provide patients with a standardized neurological assessment.

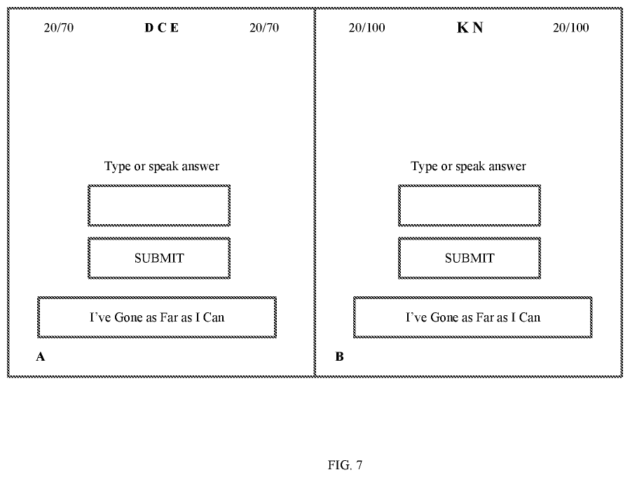

Using the blind-spot calibration, users undergo a visual acuity assessment. A row of letters is displayed at a time and the user types in what letters they see (FIG. 7A). Once the user submits their response, a new row of letters in a smaller font size is displayed (FIG. 7B). The font size of each row of letters decreases until the user cannot see any letters anymore. Each row tests visual acuity based on the Snellen Chart that ranges from 20/200 to 20/10 visual acuity. The blind spot calibration can maintain the same proportions every time the user takes the test.

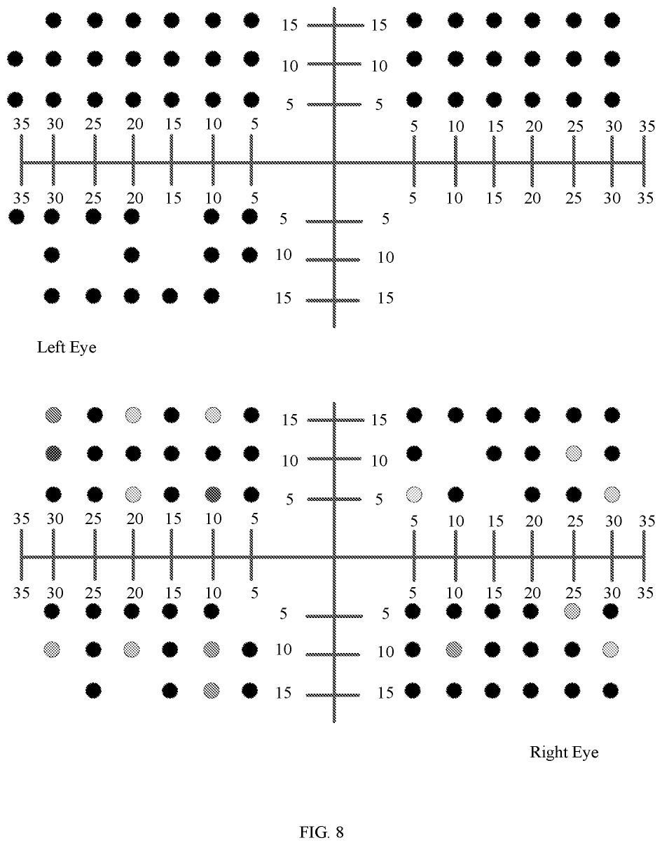

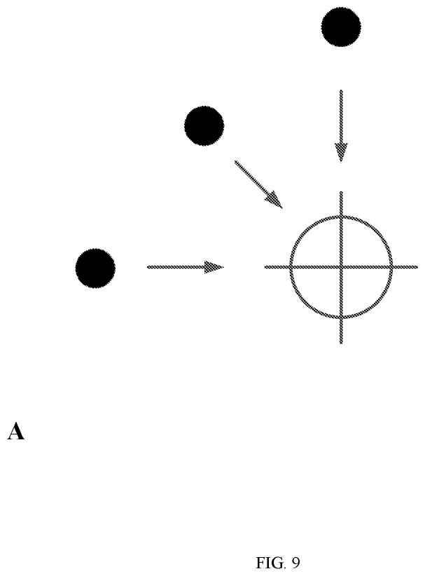

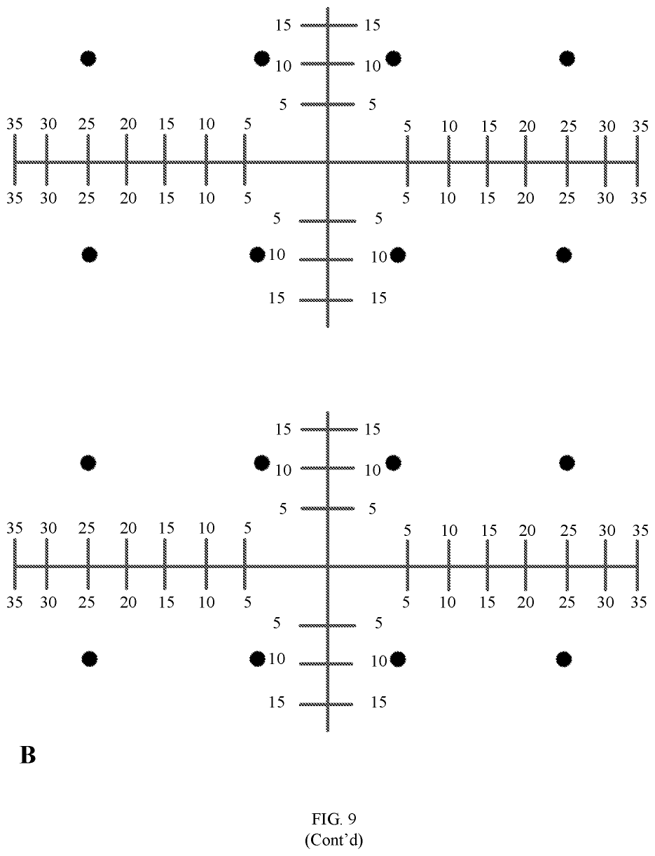

The same blind spot calibration technique can be utilized to determine the user's distance from the screen in order to administer visual field testing, assessing vision over 60 degrees horizontally and 30 degrees vertically (FIG. 8). The user closes one eye and focuses with the other eye on a crosshair at each of the 4 corners of the screen. Dots are displayed one at a time in each of the four quadrants of the user's vision with increasing color intensity. As soon as the user sees the dot, the user is instructed to press a key. The longer it takes for the user to identify the presence of a dot, the darker the dot appears. For example, FIG. 8 demonstrates a patient who is unable to see the inferonasal quadrant of his left eye, reflected by the lack of dots visualized in that quadrant during the assessment. Similarly, a kinetic visual field assessment is performed where a dot moves from one corner of the screen towards the crosshair and the user presses a key as soon as the user sees the dot (FIG. 9A). This is repeated 24 times total, 3 dots for each crosshair and there are a total of 4 crosshairs per eye. FIG. 9B demonstrates an example of the degrees of peripheral vision a user is able to see in all four quadrants of vision.

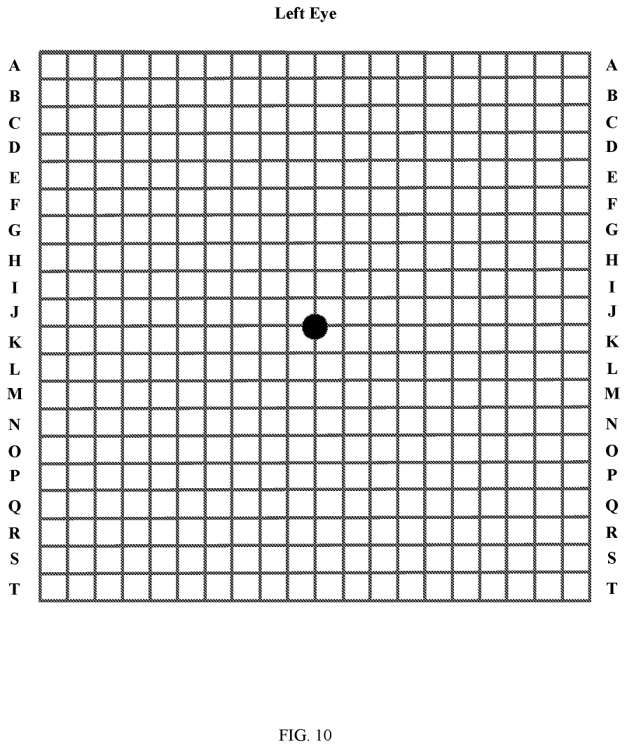

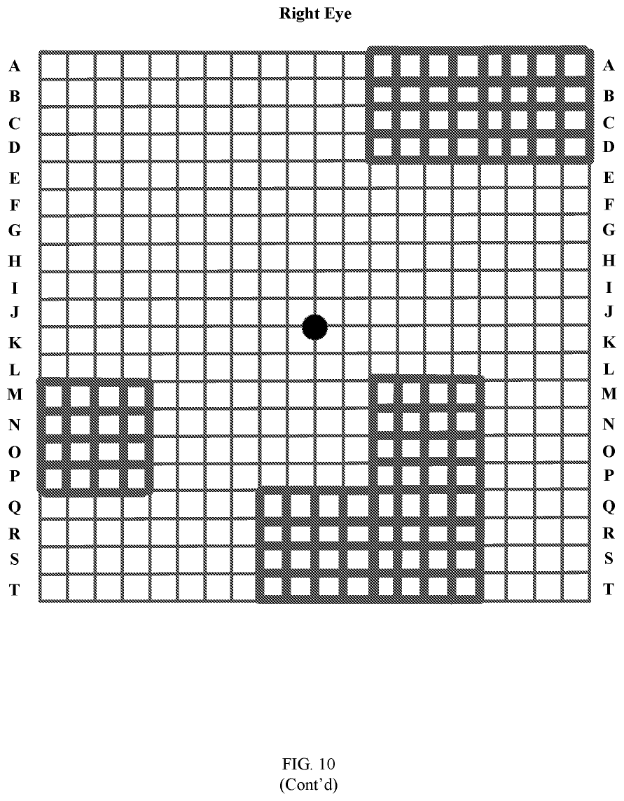

The blind spot calibration provides for a reliable Amsler grid assessment for macular degeneration. The user is asked to focus on the dot in the middle of the screen and a box of 4×4 squares is displayed at a time in a clockwise fashion around the dot. The user is asked to press a keyboard key as soon as a box of squares appears distorted or wavy. The clockwise introduction of the boxes allows the user to maintain their focus on the dot and press a key when an area appears abnormal instead of having to search for the abnormal area, which would confound the results. FIG. 10 demonstrates normal findings for the left eye, while the right eye has a few areas that appear abnormal.

The pilot study demonstrating the reliability and feasibility of the blind spot-based methods described herein involves 15 participants with a mean age of 48.7±17.1 years old, of whom 10 were females and 5 were males. The participants in this study were diagnosed with a brain tumor and were enrolled in order to undergo comprehensive cranial nerve and neuro-ophthalmic testing. The assessment findings were compared to those of in-person physical examination by the participants' treating physician. Findings are discussed from assessing 2 patients: one with severe visual complaints that serves as a positive control and one with no neurological deficits that serves as a negative control.

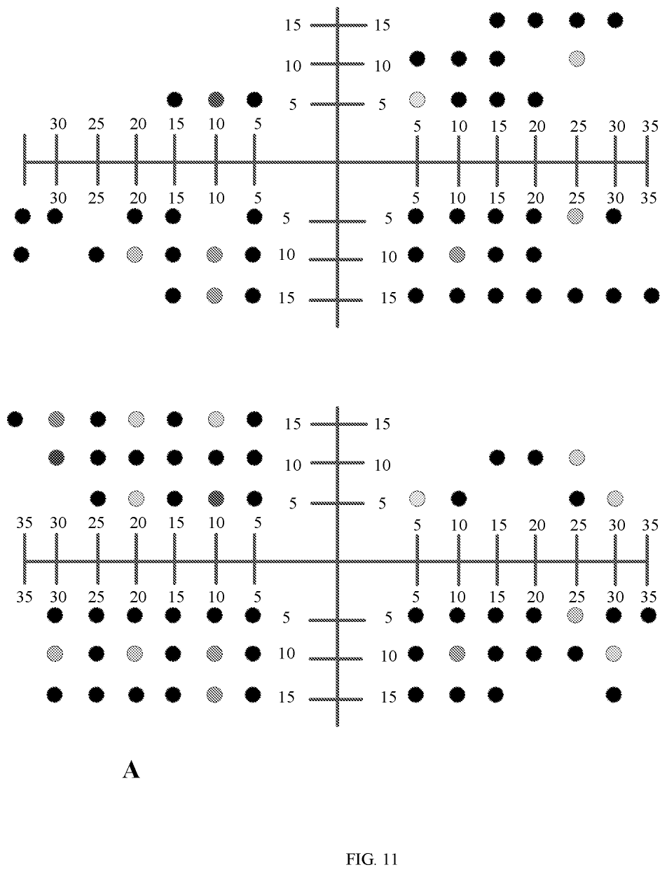

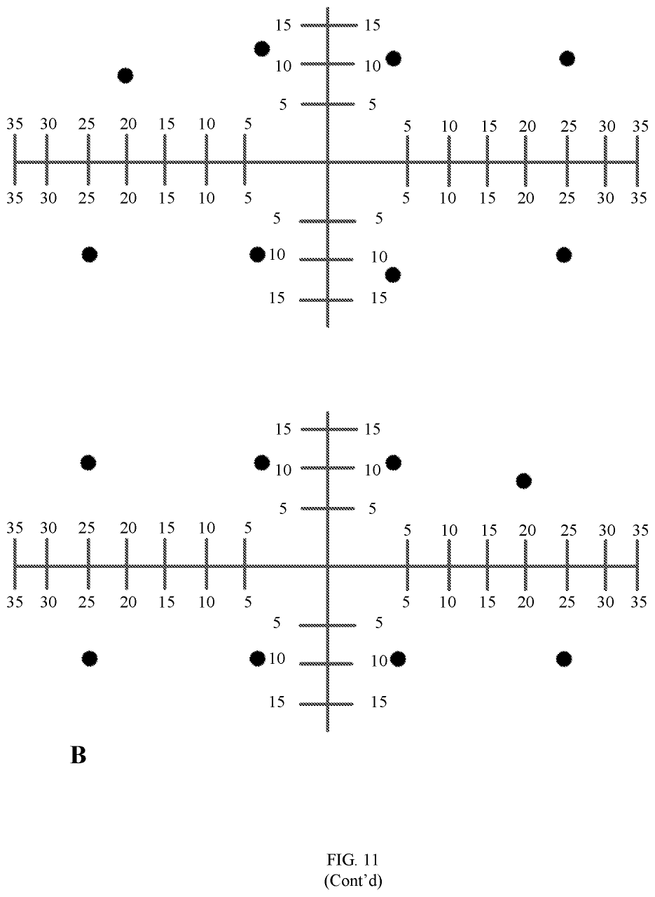

The first patient is a 53 year old male with history of pituitary adenoma that caused him to develop bilateral superior temporal visual field deficits. FIGS. 11A and 11B represent the first patient's blind spot-based static and kinetic visual field assessments respectively. These figures demonstrate constricted peripheral vision in bilateral superior outer quadrants, which is consistent with his physical examination.

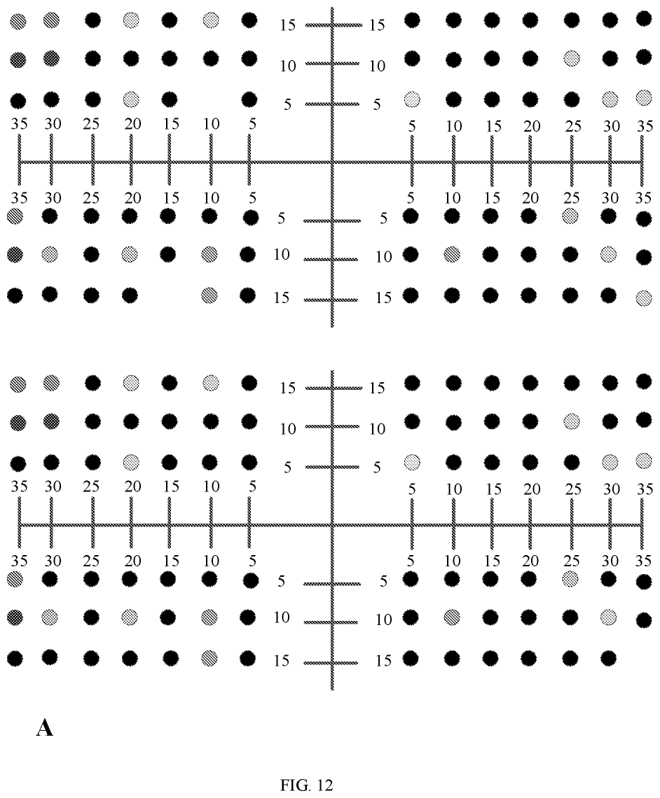

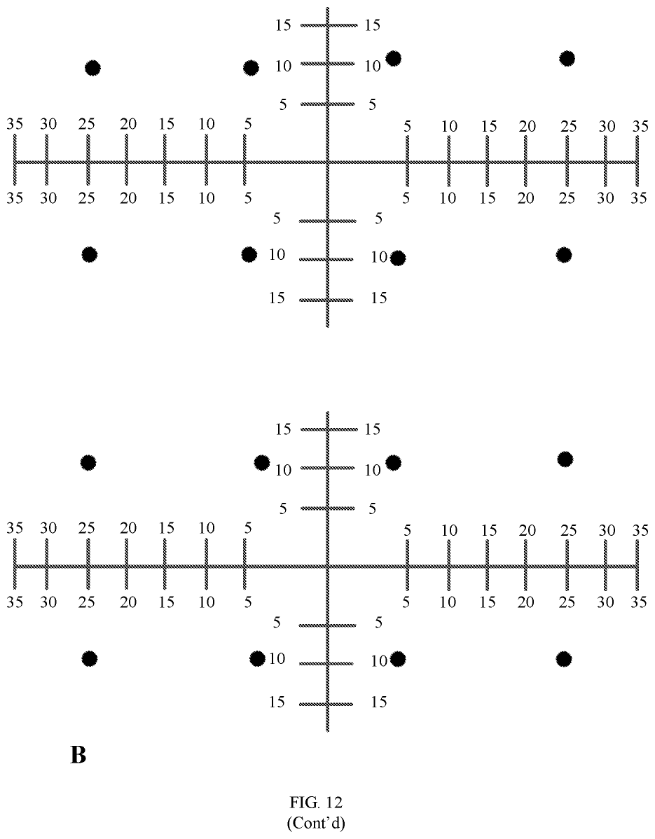

The second patient is a 49-year old female also with history of pituitary adenoma that was diagnosed incidentally without any vision complaints. Her peripheral vision was intact on physical examination, which is consistent with her static and kinetic visual fields depicted in FIG. 12.

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

1. A computer-implemented method comprising:
generating a repositionable animated object on a display screen;
receiving input from the user of the display screen when the repositionable animated object transitions from within a viewing range of the user to outside of the viewing range;
determining a position of the repositionable animated object on the display screen based on a timing of the received input; and
determining a blind spot of the user on the display screen based on the position of the repositionable animated object.

2. The computer-implemented method of claim 1, further comprising:
repositioning the repositionable animated object in a predefined direction on the display screen and at a predefined speed.

3. The computer-implemented method of claim 2, wherein the predefined direction comprises a lateral direction, wherein the blind spot comprises a lateral blind spot.

4. The computer-implemented method of claim 2, wherein the predefined direction comprises a vertical direction, wherein the blind spot comprises a vertical blind spot.

5. The computer-implemented method of claim 1, further comprising:
generating a neuro-ophthalmic test for the user based on a location of the blind spot on the display screen and a distance away from the display screen of the user.

6. The computer-implemented method of claim 5, further comprising:
generating an object size, an object color, an object luminosity, or an object graphic positioning of the neuro-ophthalmic test according to the location of the blind spot.

7. The computer-implemented method of claim 5, wherein the neuro-ophthalmic test comprises a pituitary adenoma test, a brain tumor test, a multiple sclerosis test, a neuromyelitis optica test, an optic neuritis test, an ischemic optic neuropathy test, a giant cell arteritis test, an optic neuropathy test, a retinal degenerations test, a toxic optic neuropathies and retinopathies test, or a combination thereof.

8. The computer-implemented method of claim 1, further comprising:
generating a static reference point on the display screen, wherein a repositioning of the repositionable animated object occurs in relation to the static reference point.

9. A telemedicine system, comprising:
a user device comprising at least the display screen; and
a processor configured to perform the computer-implemented method of claim 1.

10. A computer-implemented method for identifying a blind spot of a user comprising:
generating a repositionable animated object on a display screen;
receiving input from the user of the display screen when the repositionable animated object transitions from within a viewing range of the user to outside of the viewing range;
determining a position of the repositionable animated object on the display screen based on a timing of the received input; and
determining at least one blind spot for the user based on the position of the repositionable animated object.
